(19) United States
(12) Patent Application Publication
(10) Pub. No.: US 2026/0199490 A1
(43) Pub. Date: Jul. 16, 2026

(54) CORNEAL FILLER

(71) Applicant: The General Hospital Corporation, Boston, MA (US)

(72) Inventors: Xiaolei Li, Boston, MA (US); Conor L. Evans, Boston, MA (US); Reginald Birngruber, Luebeck (DE); Richard R. Anderson, Boston, MA (US); Badri Parshad, Boston, MA (US); Lara Buhl, Boston, MA (US); Gabriela Apiou, Boston, MA (US)

(21) Appl. No.: 19/135,800

(22) PCT Filed: Dec. 8, 2023
(86) PCT No.: PCT/US2023/083210

§ 371 Date: Jun. 5, 2025

Related U.S. Application Data
(60) Provisional application No. 63/386,663, filed on Dec. 8, 2022.
(60) Provisional application No. 63/580,132, filed on Sep. 1, 2023.

(51) Int. Cl. A61K 47/61; A61K 9/00; A61P 27/10

(52) U.S. Cl. CPC A61K 47/61; A61K 9/0051; A61P 27/10

(57) ABSTRACT

Disclosed are compositions, systems, and methods for treating hyperopia and presbyopia without tissue removal to increase the refractive power of the cornea. A polymer conjugate is provided, which can be used as an injectable filler material for corneal implant. In particular embodiments, the polymer conjugate comprises an aldehyde hyaluronic acid conjugated with a poly ether through a —CH═N— bond.

### CROSS-REFERENCE TO RELATED APPLICATIONS

This application is based on, claims benefit of, and claims priority to U.S. Patent Application No. 63/386,663 filed on Dec. 8, 2022, and U.S. Patent Application No. 63/580,132 filed on Sep. 1, 2023, the contents of which are hereby incorporated by reference herein in their entirety.

### STATEMENT REGARDING FEDERALLY SPONSORED RESEARCH

Not Applicable.

### BACKGROUND

The invention relates to compositions, systems, and methods for treating hyperopia and presbyopia to increase the refractive power of the cornea.

Hyperopia (farsightedness) is a common refractive error. When seeing an ophthalmologist, patients complain about blurry vision leading to a prescription of glasses or contact lenses. Even though laser-assisted procedures are standard treatments in myopia (nearsightedness) to provide patients a life without glasses, correction of hyperopia using corneal refractive surgery is still associated with serious complications, low predictability, and poor stability. Presbyopia is the irreversible loss of accommodative power leading to problems with reading and focusing objects nearby. Patients of age 50 or older gradually experience an onset of blurred near vision as one of their first symptoms. Correction of presbyopia can involve non-invasive corrections (such as reading glasses or bifocal contact lenses) and surgical treatment (such as laser refractive surgeries, corneal implants, and lens refractive surgeries), facing complications such as reduced visual acuity, glare, halos, and implant extrusion

Therefore, what is needed is compositions, systems, and methods for treating hyperopia and presbyopia without tissue removal to increase the refractive power of the cornea.

### SUMMARY OF THE DISCLOSURE

The present disclosure provides a solution to the problems associated with corneal refractive surgery for hyperopia or presbyopia. The systems and methods of the present disclosure increase the refractive power of the cornea by injecting a filler material according to the present disclosure into an intracorneal pocket. The systems and methods of the present disclosure treat hyperopia and presbyopia without tissue removal, preserve biomechanical stability, and allow for finer tuning of the refractive result. In one embodiment, the systems and methods of the present disclosure use a femtosecond laser to cut an intracorneal pocket and use transparent filler material according to the present disclosure that matches the refractive index of the cornea. The systems and methods of the present disclosure use a filler applicator to inject the filler into the intracorneal pocket.

In one aspect, the present disclosure provides a polymer conjugate, or a pharmaceutically acceptable salt thereof, comprising hyaluronic acid repeating units of formula (I) and a plurality of aldehyde units of formula (II)

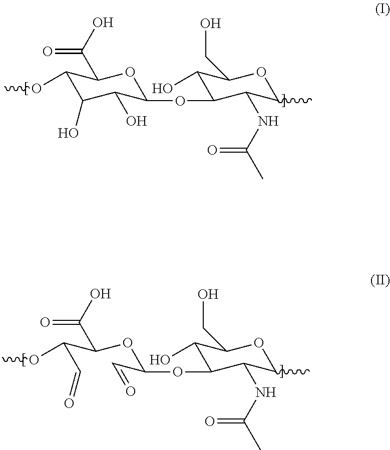

wherein at least one —C(O)H group in the plurality of aldehyde units is replaced by —CH═N—R;
and
R is a polyether group.

In some embodiments, the polyether group comprises a polyethylene glycol (PEG) group, a polyglycerol group, a pluronic group, or a combination thereof. R can have a terminal group R¹. R¹ can be, for example, H, C₁₋₄ alkyl, OH, NH₂, C₁₋₄ hydroxyalkyl, C₁₋₄ aminoalkyl, O—C₁₋₄ alkyl, or NH—C₁₋₄ alkyl.

In some embodiments, R is a 4-arm polyether group of —(CH₂CH₂O)_{y}CH₂C[CH₂(OCH₂CH₂)_{y}R¹]₃, and wherein y is 5-120. In some embodiments, R is —(CH₂CH₂O)ₜ—R¹, and wherein t is 20-500.

The polymer conjugate, or a pharmaceutically acceptable salt thereof, can have an average molecular weight of about 50 kDa to about 5000 kDa. In some embodiments, the average molecular weight is about 500 kDa to about 2000 kDa.

In another aspect, the present disclosure provides polymer conjugate, comprising an aldehyde hyaluronic acid conjugated with a polyether having a terminal amino group, whereby a —CH═N— bond is formed between an aldehyde group of the aldehyde hyaluronic acid and the terminal amino group of the polyether. The aldehyde hyaluronic acid can have an average molecular weight of about 50 kDa to about 5000 kDa. The polyether can comprise a polyethylene glycol (PEG), a polyglycerol, a pluronic polymer, or a combination thereof. The polyether can have an average molecular weight of about 1 kDa to about 50 kDa.

The polymer conjugate as described herein can be crosslinked. The polymer conjugate as described herein can have a refractive index of about 1.3 to about 1.5. The polymer conjugate as described herein can have a viscosity of about 5,000 cP to about 130,000 cP.

In another aspect, the present disclosure provides a method of preparing a polymer conjugate. The method can comprise:
reacting an aldehyde hyaluronic acid comprising hyaluronic acid repeating units of formula (I) and a plurality of aldehyde units of formula (II) with a polyether having a terminal amino group,

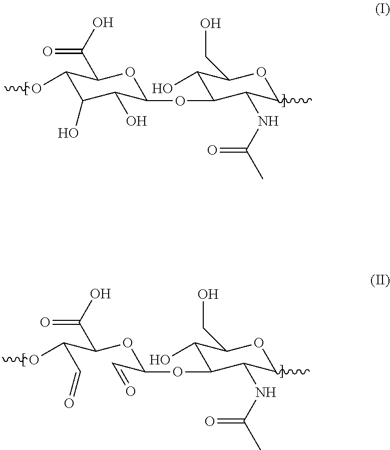

whereby at least one —C(O)H group in the plurality of aldehyde units forms a —CH═N— bond with the terminal amino group of the polyether to produce the polymer conjugate.

In another aspect, the present disclosure provides a polymer conjugate produced by the preparation method as described here.

In another aspect, the present disclosure provides an injectable filler material comprising any of the polymer conjugate as described herein, or a pharmaceutically acceptable salt thereof, and at least one additional component. The at least one additional component can comprise a liquid carrier. For example, the liquid carrier comprises water.

In another aspect, the present disclosure provides a method for treating an ametropia in a patient, the method can comprise:

injecting a filler material comprising the polymer conjugate of any one of claim 1-21 or 27-29 into the patient's cornea in an amount sufficient to steepen the anterior surface and flatten the posterior surface of the cornea to increase the refractive power of the cornea by a predetermined correction due to changing both surfaces of the cornea, wherein the polymer conjugate forms a corneal implant with a lenticular shape within the cornea.

In some embodiments, the method comprises:
making a cut in the patient's cornea with a pulsed laser to create a two-dimensional (2D) incision adjacent to and generally parallel to the anterior surface of the cornea; and
injecting the filler material into the incision.

In some embodiments, cutting the cornea comprises cutting a two-dimensional (2D) slit centered around the optical axis of the cornea, wherein the cut is made at a certain depth from an anterior surface of the cornea, and/or wherein the making the cut does not require making an opening the corneal surface and therefore does never require creating a corneal flap.

In some embodiments, the corneal implant is fixed in place by crosslinking components of the filler material to corneal tissue or by applying a crosslinking agent to an internal surface of the corneal cut.

In some embodiments, the method further comprises: chemically or enzymatically removing the corneal implant; and forming a new corneal implant by injecting a new filler material comprising another polymer conjugate as described herein. For example, the new corneal implant can have a different shape and/or a different refractive index than that of the removed corneal implant.

In yet another aspect, the present disclosure provides a method to treat presbyopia in a patient. The method comprises: creating an optical bifocality by creating a small incision (2-3 mm in diameter) in the optical center of the patient's cornea deep enough (about 50-150 μm away from the posterior surface) to selectively flatten the central part of the posterior surface of the cornea, increasing only the central refractive power by 1-3 diopters but leaving the peripheral refraction unchanged.

These and other features, aspects and advantages of various embodiments of the present disclosure will become better understood with regard to the following description, appended claims, and accompanying Figures.

### BRIEF DESCRIPTION OF THE DRAWINGS

FIG. 1A shows correction of hyperopia by injecting a biocompatible, transparent, viscous liquid filler material into a laser-generated corneal pocket to increase refractive power. The human corneal refractive index is 1.376. Representative filler materials are synthesized using hyaluronic acid (HA) and a polyethylene glycol (PEG)-amine.

FIG. 1B shows a reaction scheme according to one non-limiting embodiment of the present disclosure. Aldehyde groups of a functionalized HA polymer can react with the amino groups of a PEG-amine through a “Schiff-base” reaction to form a “Schiff-base” bond.

FIG. 1C shows ¹H-NMR analyses of HA, PEG, and HA-PEG conjugates.

FIG. 1D shows FTIR analyses of HA, PEG, and HA-PEG conjugates.

FIGS. 1E-1G show representative results of gel permeation chromatography (GPC) analyses of HA, PEG, and HA-PEG conjugates using light scattering detector (FIG. 1E), refractive index detector (FIG. 1F), and viscometer detector (FIG. 1G), respectively.

FIG. 1H shows refractive index of HA-PEG measured at 37° C. using an ABBMAT Automatic Refractometer.

FIG. 1I shows refractive correction of a hyperopic eye. Farsightedness leads to blurred vision (eyeball is too short, placing the sharp image behind the retina). After filler injection into the femtosecond laser-created corneal pocket according to the present disclosure, the refractive power increases and the image falls onto the retina. The eye is emmetropic (right-sighted).

FIG. 2 shows a refractive change after filler injection in two different depths according to the present disclosure. Stromal pockets in a depth of 120 μm lead to refractive power increases up to 8 diopters.

FIG. 3 shows a principle of presbyopia correction. Femtosecond laser-assisted, spherical corneal pockets close to the posterior curvature are established with a diameter of 2-3 mm. Injection of corneal filler leads to a central flattening of the posterior curvature and thus an increase of central refractive power according to the present disclosure.

FIG. 4 shows a refractive change after filler injection in two different depths according to the present disclosure. Stromal pockets in a depth of 120 μm lead to refractive power increases up to 8 diopters.

FIG. 5 shows that in hyperopia conditions, the image of nearby objects becomes focused behind the retina of the eye.

FIG. 6 shows a plot of the refractive index of the synthesized materials according to the present disclosure measured at varying concentrations in PBS using an Automatic Refractometer set at 33° C.

FIG. 7 shows optical coherence tomography images of rabbit cornea ex vivo after injection of the synthesized conjugates according to the present disclosure and their comparison with reported HA (2 MDa).

FIG. 8 shows the refractive index of HA-mPEG at 33° C. with a molecular weight of 1750 kDa.

FIGS. 9A and 9B show the cell viability assay results for HA-4-PEG and HA-mPEG conjugates, respectively. HA (1 MDa) was used as control for cell viability measurement.

Like reference numerals will be used to refer to like parts from Figure to Figure in the following detailed description. Any drawings herein are not shown to scale. Where dimensions are given in the text or figures, these dimensions are merely example values which could be used with one or more example implementations and do not limit the scope of the disclosed invention. Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure.

### DETAILED DESCRIPTION

Before the present invention is described in further detail, it is to be understood that the invention is not limited to the particular embodiments described. It is also to be understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It will be appreciated by those skilled in the art that while the disclosed subject matter is described herein in connection with particular embodiments and examples, the invention is not necessarily so limited, and that numerous other embodiments, examples, uses, modifications and departures from the embodiments, examples and uses are intended to be encompassed by the claims attached hereto.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “alkyl” as used herein, means a straight or branched chain saturated hydrocarbon. The alkyl can be a C₁₋₄alkyl. Representative examples of alkyl include, but are not limited to, methyl, ethyl, n-propyl, isopropyl, n-butyl, sec-butyl, isobutyl, and tert-butyl.

The representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁-C₄” or “C₁₋₄,” the members of the group that follows may have any number of carbon atoms falling within the recited range. A “C₁-C₄alkyl” or “C₁₋₄alkyl,” for example, is an alkyl group having from 1 to 4 carbon atoms, however arranged (i.e., straight chain or branched).

The term “hyaluronic acid” (HA) is understood to be an anionic, nonsulfated glycosaminoglycan composed of repeating polymeric disaccharides of D-glucuronic acid and N-acetyl-D-glucosamine linked by a glucuronidic β (1→3) bond (shown below). Such units are referred to as “hyaluronic acid repeating units.”

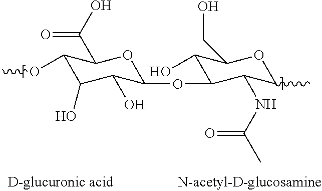

The term “aldehyde hyaluronic acid” refers to a derivatized hyaluronic acid, in which the D-glucuronic acid moieties of a plurality of hyaluronic acid repeating units are transformed into an open structure with aldehyde groups (—CHO) at C2 and C3 positions (shown below). These aldehyde-functionalized units are referred to herein as “aldehyde units.” The number of the aldehyde units can be up to 50% of total units of an aldehyde hyaluronic acid, which is the sum of the aldehyde units and the hyaluronic acid repeating units. The percentage of the aldehyde units in the total units in the aldehyde hyaluronic acid can be, for example, about 1%, about 5%, about 10%, about 20%, or about 40%.

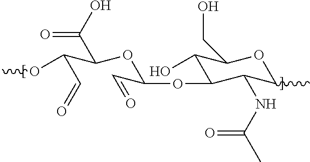

The term “pharmaceutically acceptable salt thereof” means a salt prepared by combining a compound (e.g., a polymer conjugate) as described herein with an acid whose anion, or a base whose cation, is generally considered suitable for human consumption. Pharmaceutically acceptable salts are particularly useful because of their greater aqueous solubility relative to the parent compound. For use in medicine, the salts of the present compounds are non-toxic “pharmaceutically acceptable salts”. The term “pharmaceutically acceptable salts” includes non-toxic salts of the present compounds which can be prepared by reacting the free base with a suitable organic or inorganic acid.

Suitable pharmaceutically acceptable acid addition salts of the present compounds when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids. Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, P-hydroxybutyrate, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, and undecanoate.

Further, where the present compounds carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N, N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (C₁-C₆) halides (e.g. methyl, ethyl, propyl, and butyl chlorides, bromides, and iodides), dialkyl sulfates (i.e., dimethyl, diethyl, dibutyl, and diamyl sulfates), long chain halides (e.g., decyl, lauryl, myristyl, and stearyl chlorides, bromides, and iodides), arylalkyl halides (e.g., benzyl and phenethyl bromides), and others.

As used herein, a “polyether group” mean a substituent group derived from a polyether, which is attached to the remainder of a polymeric molecule as described herein via an atom on the backbone or a side chain of the polyether.

The term “pluronic group” refers to a class of polyether groups as defined herein, in which the polyether is a triblock polymer also known as poloxamer, also referred to as poly(ethylene glycol)-block-poly(propylene glycol)-block-poly(ethylene glycol) or [poly(ethylene oxide)-block-poly(propylene oxide)-block-poly(ethylene oxide)]. For example, the poloxamer can have a structure of

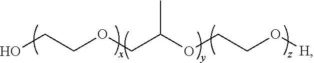

in which x, y, z are integers. The pluronic group can have a terminal group as described herein.

The term “polyglycerol group” refers to a polyether group as defined herein, in which the polyether is the product of a polymerization reaction of glycerol to form a polyglycerol, also referred to as a polyglycerine or a polyglycerin. For example, the polyglycerol can have a structure of

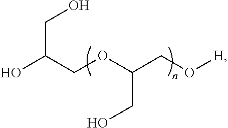

in which n is an integer. The polyglycerol group can have a terminal group as described herein.

The term “molecular weight” in this specification refers to “weight average molecular weight” (M_{w}=Σᵢ NᵢMᵢ²/Σᵢ Nᵢ Mᵢ). Although weight average molecular weight (M_{w}) can be determined in a variety of ways, with some differences in result depending upon the method employed, it is convenient to employ gel permeation chromatography.

### Composition

In one aspect, the present disclosure provides a polymer conjugate, or a pharmaceutically acceptable salt thereof, comprising hyaluronic acid repeating units of formula (I) and a plurality of aldehyde units of formula (II)

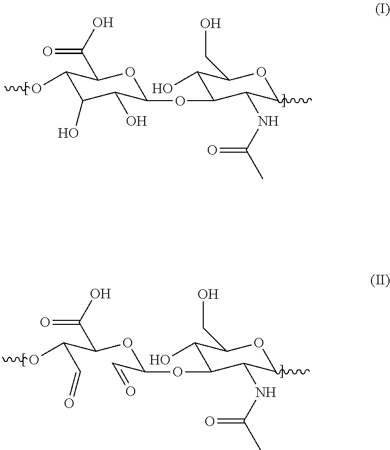

wherein
at least one —C(O)H group in the plurality of aldehyde units is replaced by —CH═N—R; and
R is a polyether group.

In some embodiments, the polymer conjugate, or a pharmaceutically acceptable salt thereof, consists of hyaluronic acid repeating units of formula (I) and the plurality of aldehyde units of formula (II), wherein at least one —C(O)H group in the plurality of aldehyde units is replaced by —CH═N—R.

The number of the aldehyde units, including those of formula (II) and those in which a —C(O)H group is replaced by —CH═N—R, can be up to 50% of the sum of the aldehyde units and the hyaluronic acid repeating units of formula (I) in the polymer conjugate. The percentage of the aldehyde units in the total units can be, for example, about 1%, about 5%, about 10%, about 20%, or about 40%.

The number of units containing —CH═N—R can be up 100% of the aldehyde units in the polymer conjugate. The percentage of the units containing —CH═N—R in the aldehyde units can be, for example, about 1%, about 5%, about 10%, about 20%, about 50%, about 80%, about 90%, about 95%, or about 99%.

In some embodiments, the polyether group comprises a polyethylene glycol (PEG) group, a polyglycerol group, a pluronic group, or a combination thereof. In some embodiments, R comprises a PEG group. The PEG group can have —CH₂CH₂O— repeating units. For example, R can include a —(CH₂CH₂O)ₘ— group, in which m is an integer. The PEG group also has a terminal group. In some embodiments, R is a PEG group.

R has a terminal group that is H or a non-H group. In some embodiments, R has a terminal group R¹ and R¹ is H, C₁₋₄ alkyl, OH, NH₂, C₁₋₄ hydroxyalkyl, C₁₋₄ aminoalkyl, O—C₁₋₄ alkyl, or NH—C₁₋₄ alkyl. In some embodiments, R¹ is H or C₁₋₄ alkyl, such as methyl or ethyl. Examples of R¹ includes, but are not limited to methyl, —CH₂CH₂OH, or —CH₂CH₂NH₂, OCH₂CH₃, and NHCH₂CH₃.

R can be a multi-arm polyether group. In some embodiments, R is a 4-arm polyether group, such as a 4-arm PEG group. For example, R can be a 4-arm polyether group of —(CH₂CH₂O)_{y}CH₂C[CH₂(OCH₂CH₂)_{y}R¹]₃, wherein y is 5-120. Nonlimiting examples of terminal group R¹ for such 4-arm polyether group include H, —OH, and —NH₂.

R can be a single-arm polyether group, such as a non-branched PEG group. In some embodiments, R is —(CH₂CH₂O)ₜ—R¹, wherein t is 20-500. Nonlimiting examples of terminal group R¹ for such polyether group include H, C₁₋₄ alkyl, —CH₂CH₂OH, and —CH₂CH₂NH₂. For example, R can be —(CH₂CH₂O)ₜ—CH₃, which is referred to as methyl-PEG or mPEG.

The polymer conjugate as described herein, or a pharmaceutically acceptable salt thereof, can have an average molecular weight of about 50 kDa to about 5000 kDa. The average molecular weight can be, for example, about 100 kDa to 5000 kDa, about 500 kDa to about 5000 kDa, about 500 kDa to about 4000 kDa, about 500 kDa to about 3000 kDa, about 500 kDa to about 2000 kDa, about 500 kDa to about 1500 kDa, or about 500 kDa to about 1000 kDa. In some embodiments, the average molecular weight is about 500 kDa to about 2000 kDa, such as about 500 kDa, about 750 kDa, about 1000 kDa, about 1500 kDa, or about 2000 kDa.

In some embodiments, the polymer conjugate, or a pharmaceutically acceptable salt thereof, is crosslinked. For example, two or more polymer conjugate molecules as described herein can be joint by a crosslinking reaction between: (a) a functional group in a hyaluronic acid repeating unit of one molecule and a functional group in a hyaluronic acid repeating unit of another molecule, (2) a functional group in a hyaluronic acid repeating unit of one molecule and a functional group in an aldehyde unit of another molecule, (3) a functional group in an aldehyde unit of one molecule and a functional group in an aldehyde unit of another molecule, (4) a terminal R¹ group in an aldehyde unit of one molecule and a functional group from either a hyaluronic acid repeating unit or an aldehyde unit of another molecule, or (5) a terminal R¹ group in an aldehyde unit of one molecule and a terminal R¹ group in an aldehyde unit of another molecule.

The polymer conjugate as described herein, or a pharmaceutically acceptable salt thereof, can be transparent material useful for implant to treat a human condition, such as ametropia. The polymer conjugate can have desired properties, including for example refractive index and viscosity, for such purposes. In some embodiments, the polymer conjugate, or a pharmaceutically acceptable salt thereof, has a refractive index of about 1.3 to about 1.5. The refractive index of the present polymer conjugate can be, for example, about 1.30 to about 1.45, about 1.35 to about 1.45, about 1.35 to about 1.40, about 1.35 to about 1.39, about 1.35 to about 1.38, about 1.36 to about 1.39, or about 1.36 to about 1.38. In some embodiments, the refractive index of the present polymer conjugate can be close to, or the same as, human corneal refractive index, 1.376. For example, the refractive index of the present polymer conjugate can be 1.370, 1.372, 1.374, 1.376, 1.378, or 1.380.

In some embodiments, the polymer conjugate, or a pharmaceutically acceptable salt thereof, has a viscosity of about 5,000 cP (centipoise) to about 130,000 cP. For example, the viscosity can be about 7,500 cP to about 125,000 cP, about 7,500 cP to about 100,000, about 10,000 cP about 120,000 cP, about 20,000 cP about 120,000 cP, or about 50,000 cP about 120,000 cP. In some embodiments, the polymer conjugate can be a non-newtonian fluid whose viscosity is force- or pressure-dependent.

In another aspect, the present disclosure provides a polymer conjugate, comprising an aldehyde hyaluronic acid conjugated with a polyether having a terminal amino group, whereby a —CH═N— bond is formed between an aldehyde group of the aldehyde hyaluronic acid and the terminal amino group of the polyether.

As described herein, the aldehyde hyaluronic acid has aldehyde-functionalized units, such as the aldehyde units of formula (II). The number of the aldehyde units can be up to 50% of total units of the aldehyde hyaluronic acid, including, for example, about 1%, about 5%, about 10%, about 20%, or about 40%.

In some embodiments, the aldehyde hyaluronic acid has an average molecular weight of about 50 kDa to about 5000 kDa, including, but not limited to, about 100 kDa to 5000 kDa, about 500 kDa to about 5000 kDa, about 500 kDa to about 4000 kDa, about 500 kDa to about 3000 kDa, about 500 kDa to about 2000 kDa, about 500 kDa to about 1500 kDa, or about 500 kDa to about 1000 kDa. In some embodiments, the average molecular weight of the aldehyde hyaluronic acid is about 500 kDa to about 2000 kDa, such as about 500 kDa, about 750 kDa, about 1000 kDa, about 1500 kDa, or about 2000 kDa.

The polyether can comprise a polyethylene glycol (PEG), a polyglycerol, a pluronic polymer, or a combination thereof, which is functionalized with a terminal amino group (—NH₂). In some embodiments, the polyether having a terminal amino group has a formula of R—NH₂, in which R is a polyether group as defined herein. R can have a terminal group R¹ as defined herein. In some embodiments, R is a PEG group. As nonlimiting examples, R can be a 4-arm polyether group of —(CH₂CH₂O)_{y}CH₂C[CH₂(OCH₂CH₂)_{y}R¹]₃, wherein y is 5-120; or R can be —(CH₂CH₂O) t-R¹, wherein t is 20-500.

In some embodiments, the polyether has an average molecular weight of about 1 kDa to about 50 kDa, including, but not limited to, about 1 kD to about 40 kDa, about 1 kD to about 30 kDa, about 1 kD to about 20 kDa, about 1 kD to about 15 kDa, about 1 kDa to about 10 kDa, about 2 kDa to about 30 kDa, about 2 kDa to about 20 kDa, about 2 kDa to about 10 kDa, about 2 kDa to about 5 kDa, about 5 kDa to about 30 kDa, about 5 kDa to about 20 kDa, and about 5 kDa to about 15 kDa. In some embodiments, the average molecular weight of the polyether is about 2 kDa, about 5 kDa, about 10 kDa, about 15 kDa, about 20 kDa, about 25 kDa, about 30 kDa, about 35 kDa, about 40 kDa, or about 45 kDa.

In some embodiments, the polymer conjugate is crosslinked, for example, by a crosslinking reaction between two or more polymer conjugate molecules as described herein.

The polymer conjugate can have a refractive index of about 1.3 to about 1.5, including, but not limited about 1.30 to about 1.45, about 1.35 to about 1.45, and about 1.35 to about 1.40. In some embodiments, the refractive index of the polymer conjugate is close to, or the same as, human corneal refractive index, 1.376. For example, the refractive index of the polymer conjugate can be 1.370, 1.372, 1.374, 1.376, 1.378, or 1.380.

The aldehyde hyaluronic acid and polyether conjugate can have a viscosity of about 5,000 cP to about 130,000 cP. For example, the viscosity can be about 7,500 cP to about 125,000 cP, about 7,500 cP to about 100,000, about 10,000 cP about 120,000 cP, about 20,000 cP about 120,000 cP, or about 50,000 cP about 120,000 cP.

### Method of Preparation

In another aspect, the present disclosure provides a method of preparing a polymer conjugate, the method comprising:
reacting an aldehyde hyaluronic acid comprising hyaluronic acid repeating units of formula (I) and a plurality of aldehyde units of formula (II) with a polyether having a terminal amino group,

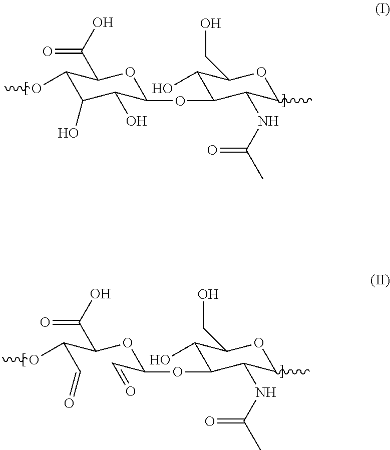

whereby at least one —C(O)H group in the plurality of aldehyde units forms a —CH═N— bond with the terminal amino group of the polyether to produce the polymer conjugate.

As described herein, the aldehyde hyaluronic acid for the present preparation method has aldehyde-functionalized units of formula (II). The number of the aldehyde units can be up to 50% of total units of the aldehyde hyaluronic acid, including, for example, about 1%, about 5%, about 10%, about 20%, or about 40%.

In some embodiments, the aldehyde hyaluronic acid for the present preparation method has an average molecular weight of about 50 kDa to about 5000 kDa, including, but not limited to, about 100 kDa to 5000 kDa, about 500 kDa to about 5000 kDa, about 500 kDa to about 4000 kDa, about 500 kDa to about 3000 kDa, about 500 kDa to about 2000 kDa, about 500 kDa to about 1500 kDa, or about 500 kDa to about 1000 kDa. In some embodiments, the average molecular weight of the aldehyde hyaluronic acid is about 500 kDa to about 2000 kDa, such as about 500 kDa, about 750 kDa, about 1000 kDa, about 1500 kDa, or about 2000 kDa.

The polyether for the present preparation method can comprise a polyethylene glycol (PEG), a polyglycerol, a pluronic polymer, or a combination thereof, which is functionalized with a terminal amino group (—NH₂). In some embodiments, the polyether is having a terminal amino group has a formula of R—NH₂, in which R is a polyether group as defined herein. R can have a terminal group R¹ as defined herein. In some embodiments, R is a PEG group. As nonlimiting examples, R can be a 4-arm polyether group of —(CH₂CH₂O)_{y}CH₂C[CH₂(OCH₂CH₂)_{y}R¹]₃, wherein y is 5-120; or R can be —(CH₂CH₂O)ₜ—R¹, wherein t is 20-500.

In some embodiments, the polyether for the present preparation method has an average molecular weight of about 1 kDa to about 50 kDa, including, but not limited to, about 1 kD to about 40 kDa, about 1 kD to about 30 kDa, about 1 kD to about 20 kDa, about 1 kD to about 15 kDa, about 1 kDa to about 10 kDa, about 2 kDa to about 30 kDa, about 2 kDa to about 20 kDa, about 2 kDa to about 10 kDa, and about 2 kDa to about 5 kDa, about 5 kDa to about 30 kDa, about 5 kDa to about 20 kDa, and about 5 kDa to about 15 kDa. In some embodiments, the average molecular weight of the polyether is about 2 kDa, about 5 kDa, about 10 kDa, about 15 kDa, about 20 kDa, about 25 kDa, about 30 kDa, about 35 kDa, about 40 kDa, or about 45 kDa.

In some embodiments, the present preparation method further comprises crosslinking the polymer conjugate to form a crosslinked polymer conjugate. The crosslinking process may include one or more crosslinking reactions as described herein.

The reaction between the aldehyde hyaluronic acid and the polyether having a terminal amino group can be carried out using known techniques and instruments. In some embodiments, the aldehyde hyaluronic acid and the polyether are dissolved in a buffer (e.g., phosphate buffer) and the reaction is carried out at a temperature of 30-50° C. for a duration of 20-60 hours under constant stirring.

In another aspect, the present disclosure provides a polymer conjugate produced by the preparation method as described herein.

The polymer conjugate produced by the present method can have a refractive index of about 1.3 to about 1.5, including, but not limited about 1.30 to about 1.45, about 1.35 to about 1.45, and about 1.35 to about 1.40. In some embodiments, the refractive index of the produced polymer conjugate is close to, or the same as, human corneal refractive index, 1.376. For example, the refractive index of the polymer conjugate can be 1.370, 1.372, 1.374, 1.376, 1.378, or 1.380.

The polymer conjugate produced by the present method can have a viscosity of about 5,000 cP to about 130,000 cP. For example, the viscosity can be about 7,500 cP to about 125,000 cP, about 7,500 cP to about 100,000, about 10,000 cP about 120,000 cP, about 20,000 cP about 120,000 cP, or about 50,000 cP about 120,000 cP.

### Injectable Filler Material

In another aspect, the present disclosure provides an injectable filler material comprising the polymer conjugate as described herein, or a pharmaceutically acceptable salt thereof, and at least one additional component. In various embodiments, the filler material is useful as a corneal filler or corneal implant material.

The present injectable corneal filler material for ophthalmic applications can be used to adjust the refractive power of the corneal for correction of ametropia. In some embodiments, the filler material is injected into a pocket created in the cornea to change the refractive power to correct ametropia (e.g., hyperopia or presbyopia). In particular embodiments, the material injected into the cornea are transparent for visible light and have a predetermined index of refraction. For example, the index of refraction of the material can match that of the corneal stroma in order to avoid optical inhomogeneities in the cornea, leading to light reflections and scattering in the cornea. The index of refraction can also be adjusted to be higher or lower than that of the surrounding cornea stroma to create additional refractive power due to the lenticular shape of the injected filler material. Further, the injected filler material can change the shape of the cornea to correct the refractive error of the eye. In another embodiment, the index of refraction of the material can be set higher or lower than the final intended index of refraction such that addition or removal of at least one additional component can be used to adjust the index of refraction to a desired value.

The filler material can have additional advantageous properties, including: flowability for injection through a thin, high gauge (e.g., 32 gauge or smaller) needle into a corneal pocket; high molecular weight to be long term retained within the corneal pocket; resistance to endogenous enzymatic and thermal degradation; and being degradable (e.g., via an injected enzyme or agent) so that the filler material can be changed or removed as needed. In addition to flowability, the filler materials may have a viscosity that is high enough to be stable within the corneal environment and able to withstand forces due to eye pressure, eye movement, blinking, and the rigidity of the cornea itself. For example, the viscosity of the filler can be about 5,000 cP to about 130,000 cP. This includes, but is not limited to, a viscosity of about 7,500 cP to about 125,000 cP, about 7,500 cP to about 100,000, about 10,000 cP about 120,000 cP, about 20,000 cP about 120,000 cP, or about 50,000 cP about 120,000 cP.

The filler materials can be biologically stable within the cornea, being resistant to enzymatic degradation. The fillers can be biocompatible, non-antigenic (although the cornea is immune privileged, strongly antigenic filler materials should be avoided), non-toxic, and non-irritating. The fillers can at the same time be permeable enough to permit oxygen and nutrient transfer through the filler and cornea. A thin layer of live cornea overlying the filler can receive much of its nutrients and eliminates much of its waste products of metabolism, by diffusion of these substances through the underlying filler. When placed close to the endothelial inner surface of cornea, fillers can be less permeable, or even impermeable.

The corneal filler material is transparent so as not to interfere with or reduce light transmission through the cornea. In addition, the present filler material can avoid light scattering and tissue stimulation.

Acceptable refractive indices of the filler materials can be similar, lower, or higher than that of the cornea (n=1.376), for example 1.300 to 1.500. The refractive index of the filler material, as well as the geometrical and volumetric effects referred to above, also affects the refractive power of the eye. Refractive index of the filler material is therefore an important factor in filler material design. The refractive index of many fillers can be tuned by changes in filler composition, e.g. the amount of solid material, crosslinking, and water binding by filler materials can affects refractive index. By using a filler material with a refractive index higher than that of the cornea, an even greater refractive correction can be achieved, e.g., up to about a total of about 30 diopters. For example, for an corneal implant, if the refractive index of the filler material is higher than that of the cornea (e.g., about 1.4 to about 1.6) and the amount of filler material is sufficient to flatten the interior surface of the cornea, then the lenticular-shaped filler material itself can add an additional refractive change of about 10 to 25 diopters, which, when added to the change caused by the flattening, provides an overall refractive correction of about 14 to 30 diopters.

Water exchange can take place between the corneal implant and the surrounding cornea after implantation, proportional to the amount of free water (vs. bound water) in the implant that is available for exchange. The amount of bound water in the cornea is very small, making up less than 10% of the total corneal water, and so nearly all water is available for diffusion and possible interaction with the free water in the implant. A stable filler material must have free water activity similar to that of cornea water. Alternatively, the implant can have minimal free water and/or be hydrophobic material with low water content, so as to reduce water exchange with the cornea, e.g., less than 10%, less than 5%, or less than 1%. In a nonlimiting embodiment, the amount of water in the injected material can be adjusted to compensate for any loss of water from the injected material into the surrounding corneal stroma.

The filler materials can be liquids or gels, e.g., hydrogels. The present polymer conjugate may need to be mixed with a liquid and/or other ingredients to form the injectable filler material. In some embodiments, the at least one additional component of injectable filler material comprises a liquid carrier. In some embodiments, the liquid carrier includes water. For example, the polymer conjugate is in dry form (e.g., a solid or semi-solid) and is dissolved or reconstituted in water or an aqueous solution to form the injectable filler material.

In some embodiments, the polymer conjugate comprises an aldehyde hyaluronic acid conjugated with a polyether having a terminal amino group as described herein. In some embodiments, the polymer conjugate is produced by the preparation method as described herein.

### Method of Use

In another aspect, the present disclosure provides a method for treating an ametropia in a patient, the method comprising:
injecting a filler material comprising the polymer conjugate as described herein into the patient's cornea in an amount sufficient to steepen the anterior surface and flatten the posterior surface of the cornea to increase the refractive power of the cornea by a predetermined correction due to changing both surfaces of the cornea,
wherein the polymer conjugate forms a corneal implant with a lenticular shape within the cornea.

In some embodiments, a two-dimensional incision is created below the corneal surface, within the stroma of the cornea with a pulsed laser. The two-dimensional (2D) incision is adjacent to and generally parallel to the anterior surface of the cornea. The method may further comprise injecting the filler material into a centrally located small and deep incision to induce a central flattening of the posterior surface of the cornea creating bifocality as treatment of presbyopia. Following the injection of the filler material into the corneal incision, the polymer conjugate may form a corneal implant with a lenticular shape. This lenticular implant may steepen the anterior surface and flatten the posterior surface of the cornea, both increasing its refractive power.

In general, to perform this correction, a two-dimensional (“2D”) cut or incision is created inside the cornea, within the stroma of the cornea, e.g., with a pulsed laser. Such lasers have been used in eye surgery before and are well described (e.g., LASIK); the laser is typically a femtosecond, picosecond or nanosecond pulsed near-infrared or ultraviolet laser. In the methods described herein a small, or no, incision through the cornea surface is needed, and no corneal flap is ever created. In this regard, the cornea remains mechanically stable and more intact than after LASIK, and much more intact than after corneal ring placement. The laser cutting is performed either superficially at a depth between 100 μm to 350 μm for hyperopia correction, or at a depth, ranging from about 250 to 450 μm deep, e.g., 300 to 450 μm or 350 to 400 μm deep (measured from the anterior or outer surface of the cornea), and extending over a circular or oval-shaped region that extends over much, if not all, of the central cornea responsible for refraction of light of the eye. The diameter of this intra-cornea cutting is approximately 2 to 9 mm, e.g., 2 to 4 mm for bifocality, or 4 to 8 mm, or 5 to 6 or 7 mm. A transparent cornea filler material is injected into the corneal incision (two-dimensional cut) created by the pulsed laser cutting. A fine needle is used, typically inserted into the edge of the cutting space, with very minimal trauma to the cornea. A volume of filler is added that is sufficient to steepen the outer (anterior) and flatten the inner (posterior) corneal surface. The filler increases the curvature of the anterior surface and decreases the curvature on the posterior surface of the cornea a controlled manner, resulting in appropriate correction of hyperopia. In addition, a second, more superficial cut can be made that when injected with a filler material (either the same or different from the filler material injected into the deep slit) increases the curvature of the outer (anterior) surface of the cornea to produce a higher overall vision correction.

By utilizing a deep corneal cut it is possible to fine-tune, and precisely increase the refractive power of the cornea by up to 4 to 5 diopters. Because of the “quietness” of the inner corneal surface a more sophisticated reshaping of that surface is possible and is quite stable in the long term. A typical example of such a sophisticated re-shaping is the correction of presbyopia, where a bi-focal shape of the inner corneal surface is required. This bi-focal shaping can be achieved either by injecting a sufficient amount of filler material into a small and deep corneal cut or by injecting a filler material into a deep annular or ring-shaped corneal cut. An intentional modification of the inner surface of the cornea for hyperopia treatment up to 4.5 or 5 diopters or a combination of anterior and posterior surface modification is particularly advantageous. In addition, by using a filler material with a refractive index higher than that of the cornea, an even greater refractive correction can be achieved, e.g., up to about a total of about 30 diopters. For example, for the deep cut, if the refractive index of the filler material is higher than that of the cornea, e.g., greater than 1.4, such as about 1.4 to 1.6, e.g., about 1.5, e.g., 1.45 to 1.55, or 1.53 to 1.58, and the amount of filler material is sufficient to flatten the interior surface of the cornea, then the lenticular-shaped filler material itself can add an additional refractive change of about 10 to 25 diopters, e.g., about 12 to 23 diopters, 14 to 21 diopters, 15 to 20 diopters, or 16 to 18 diopters, which when added to the change caused by the flattening, provides an overall refractive correction of about 14 to 30 diopters, e.g., 16 to 28 diopters, 18 to 26 diopters, 20 to 24 diopters. In some circumstances, such a correction can avoid the need to implant in inter-ocular lens (“IOL”) after surgery to remove a cataract (a cloudy lens).

The methods described herein can be adapted for correction of hyperopia, presbyopia, and/or astigmatism. The slit may be a circularly shaped 2D slit to correct spherical hyperopia. The slit may be a non-circularly oval shaped 2D slit to correct hyperopic astigmatism. If astigmatism is also to be corrected, the shape of the laser cutting can be made ovoid rather than circular, with the long axis of the oval oriented across the axis of the astigmatism. Thus, both astigmatism and hyperopia can be simultaneously corrected.

Detailed optical analysis shows that the total corneal refraction consists of two refractive parts, the anterior refraction (between the air and superior or anterior corneal surface) which is about 48 diopters (dpt) and the posterior part (between the posterior or inner corneal surface and the anterior chamber fluid surface) that is about −5 dpt, resulting in the total corneal refraction of about 43 dpt. If the filler material is injected into a deep cut, the posterior or inner surface of the cornea flattens, which leads to an increased refractive power of the cornea. In addition, if one adds a superficial cut and injects a filler material, then the anterior or outer surface of the cornea bulks out and causes an increase in the refractive power of the cornea. Therefore, according to the present disclosure, both refractive changes need to be taken into account.

In some embodiments, the method further comprises solidifying the filler material after injecting the filler composition to form a solid or semi-solid corneal implant. In some embodiments, solidifying the filler material comprises crosslinking the filler material. The crosslinking of the filler material may occur post-implant.

In some embodiments, the method may comprise fixing the corneal implant in place within the corneal cut. The corneal implant may be fixed in place by crosslinking components of the filler material to corneal tissue or by applying a crosslinking agent to an internal surface of the corneal cut. The crosslinking may occur post-implant.

In some embodiments, the method further comprises: chemically or enzymatically removing the corneal implant, and forming a new corneal implant by injecting a new filler material comprising a new polymer conjugate. The new corneal implant may have a different shape and/or a different refractive index than that of the removed corneal implant.

The present disclosure provides a solution to the problems associated with corneal refractive surgery for hyperopia. The disclosed method can increase the refractive power of the cornea by injecting filler material into an intracorneal pocket. The disclosed method can treat hyperopia and presbyopia without tissue removal, preserves biomechanical stability, and allows for finer tuning of the refractive result. In some embodiments, the disclosed method uses a femtosecond laser to cut an intracorneal pocket using a clinically approved femtosecond laser technology. The disclosed method uses transparent filler material that matches the refractive index of the cornea. The disclosed method can use a filler applicator to inject the filler into the intracorneal pocket. Anterior-segment optical coherence tomography (OCT) is used to measure the change in total corneal power using an approved OCT technology.

Additional elements of corneal implant for treating hyperopia or presbyopia are described in U.S. Pat. Nos. 10,736,731 and 11,076,994, which are incorporated by reference herein in their entirety.

### EXAMPLES

The following Examples are provided to demonstrate and further illustrate certain embodiments and aspects of the present invention and are not to be construed as limiting the scope of the invention. The statements provided in the Examples are presented without being bound by theory.

### Example 1

### Overview of Example 1

Hyperopia (farsightedness) is a common refractive error with an overall prevalence of 10% worldwide [Ref. 1.1], for which we are developing an effective new solution. When seeing an ophthalmologist, patients complain about blurry vision leading to a prescription of glasses or contact lenses. Even though laser-assisted procedures are standard treatments in myopia (nearsightedness) to provide patients a life without glasses, correction of hyperopia is still associated with serious complications, low predictability and poor stability. A viable clinical application is still missing.

Presbyopia is the irreversible loss of accommodative power leading to problems with reading and focusing objects nearby. By the age of 50, the capability of the eye to increase the refractive power settles at around 0.50 diopters (compared to 7-10 diopters in the first two decades of life) [Ref. 1.5]. Gradually, patients experience an onset of blurred near vision as one of their first symptoms. Possible non-invasive corrections are reading glasses or bifocal contact lenses. Surgical treatment options range from laser refractive surgeries and corneal implants to lens refractive surgeries, facing complications such as reduced visual acuity [Ref. 1.7], glare, halos [Ref. 1.8] and implant extrusion.

### Summary of Example 1

Described herein are HA-PEG conjugates and products that were investigated by Nuclear Magnetic Resonance (NMR), Fourier-transform Infrared Spectroscopy (FTIR), and Gel Permeation Chromatography (GPC) to confirm successful conjugation. It was also confirmed that these HA-PEG conjugates can be chemically tuned to achieve a wide range of refractive index values and can serve as corneal filler materials for correction of hyperopia presbyopia and other ametropies.

Disclosed herein are embodiments of an injectable corneal filler material for ophthalmic applications that can be used to adjust the refractive power of the corneal for correction of ametropia. The filler material is injected into a pocket created in the cornea to change the refractive power to correct ametropia (e.g., hyperopia or presbyopia). The material injected into the cornea must be transparent to visible light and have a predetermined index of refraction.

Specifically, in one embodiment, the index of refraction of the material should match that of the corneal stroma in order to avoid optical inhomogeneities in the cornea, leading to light reflections and light scattering. The injected filler material should only change the shape of the cornea to correct the refractive error of the eye (e.g., hyperopia or presbyopia).

In another embodiment, the index of refraction can be adjusted to be higher or lower than that of the surrounding cornea stroma to create additional refractive power due to the lenticular shape of the injected filler material.

There are additional material properties that must be achieved for corneal filler applications. The material must be flowable, such that it can be injected through a high gauge (thin) needle into the created corneal pocket. Further, the material must be high molecular weight so that it is long term retained within the corneal pocket. The material must be resistant to endogenous enzymatic and thermal degradation so that that material remains within the pocket. Importantly, in case the individual needs to have the corneal filler procedure re-done or removed, the material should be degradable (via an injected enzyme or material) so that the corneal filler can be changed or altered in the future.

In this study, chemically conjugated polymers based on hyaluronic acid (HA) and polyethylene glycol (PEG) were developed for ophthalmic applications. These HA-PEG conjugates were chemically tuned to achieve a wide range of refractive index values. The HA-PEG conjugates can be used as corneal filler materials for correction of hyperopia, presbyopia and other ametropias. The HA-PEG has high transparency in the visible spectrum and can be made flowable depending on the cross-linking used. The material is resistant to enzymatic and thermal degradation present in the cornea, but can be enzymatically degraded through the introduction of hyaluronidase.

### Methods

In a representative process, to make the HA-PEG conjugate, a concentration of 3% (w/v) aldehyde HA (50 kDa) was dissolved in phosphate buffer (PBS) and mixed with 10% (w/v, in PBS) 4-arm amino-terminated PEG (2 kDa) at a volume ratio of 1/6. The reaction was carried out under constant stirring for 20 hours at 40° C. Various percentages and ratios of HA and PEG can be used, as per molar weight and solubility of both HA and PEG. In some processes, both reactants were dissolved in minimum amount for water, mixed, and then stir at 45° C. for 48 h. The crude product was purified by Amicon 10 kDa cutoff centrifugal filter (4000 rpm, 30 minutes) and then underwent dialysis against deionized water for 2 days (molecular weight cutoff 10 kDa) to remove residual unreacted PEG. The resulting HA-PEG conjugate was collected as solid powder after lyophilization. This solid powder is thought to have a long shelf life and can be readily reconstituted to form an injectable corneal filler material.

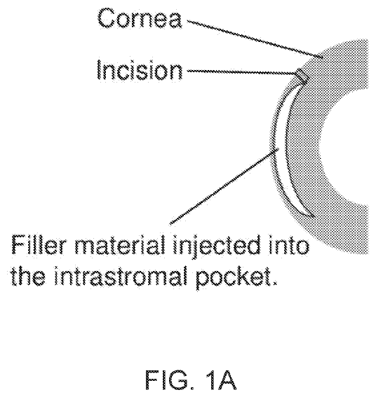

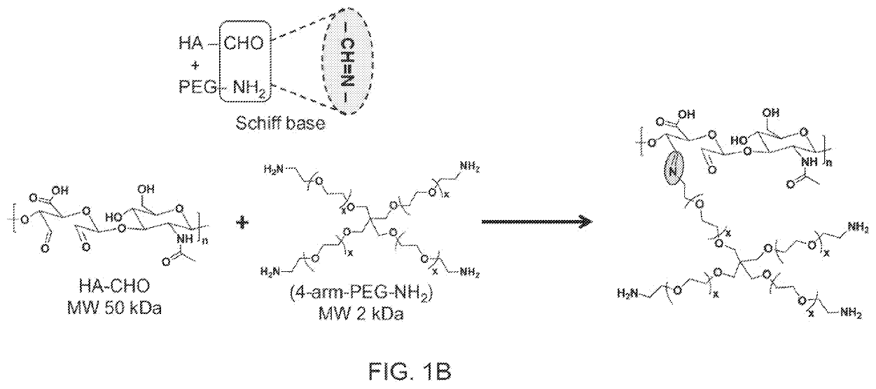

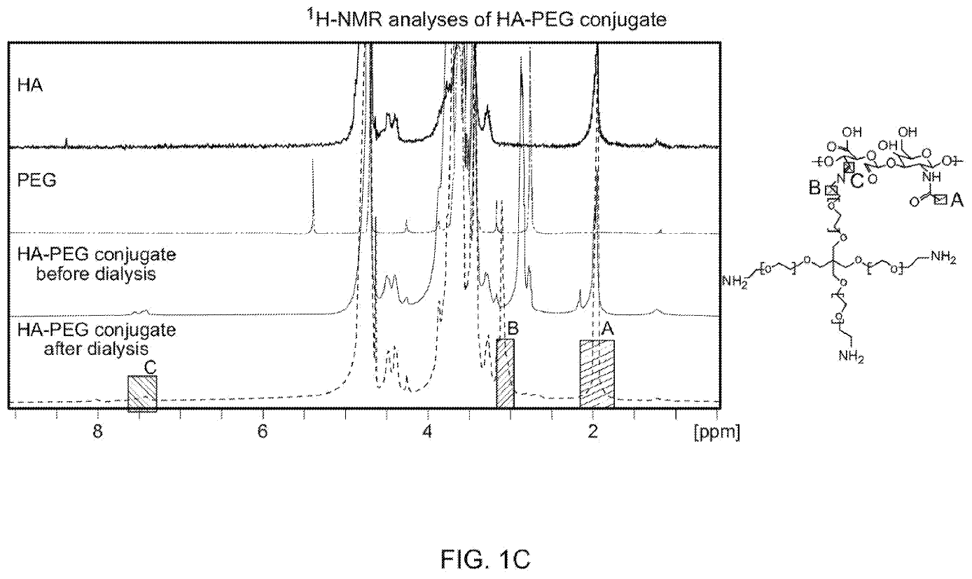

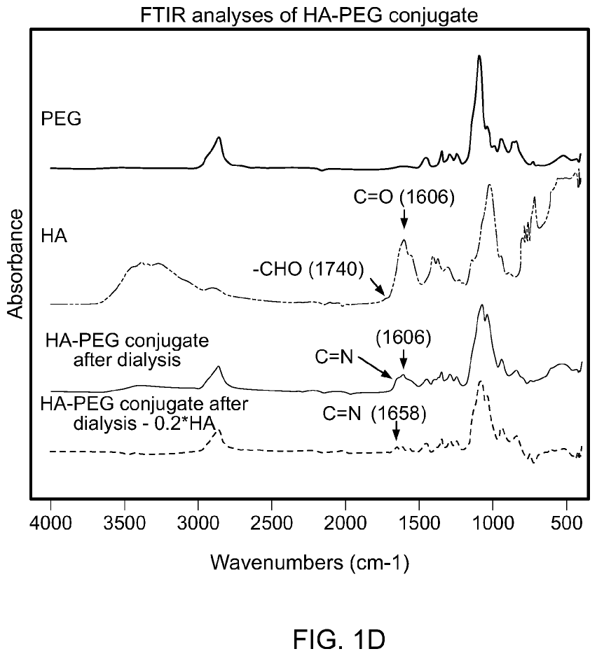

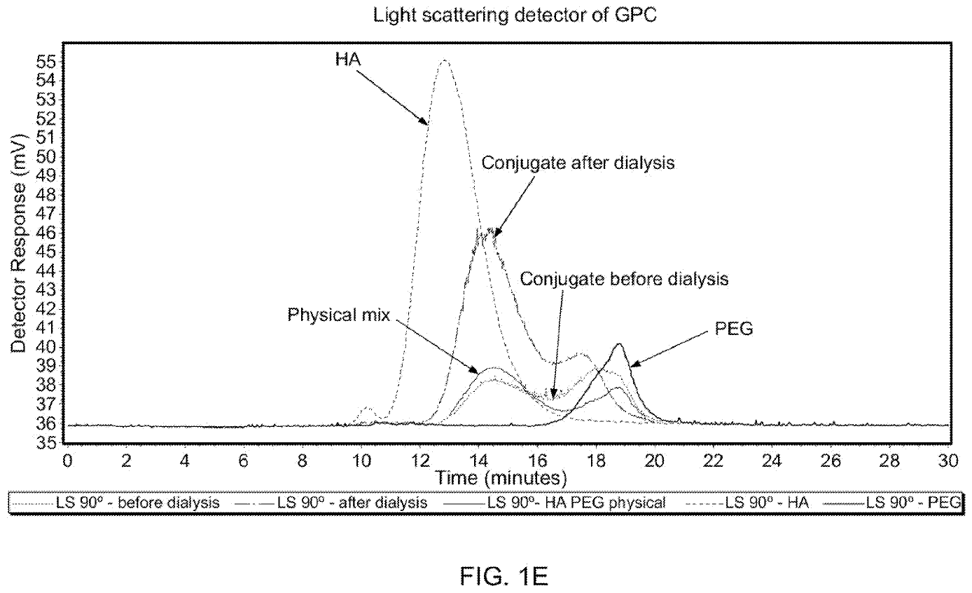

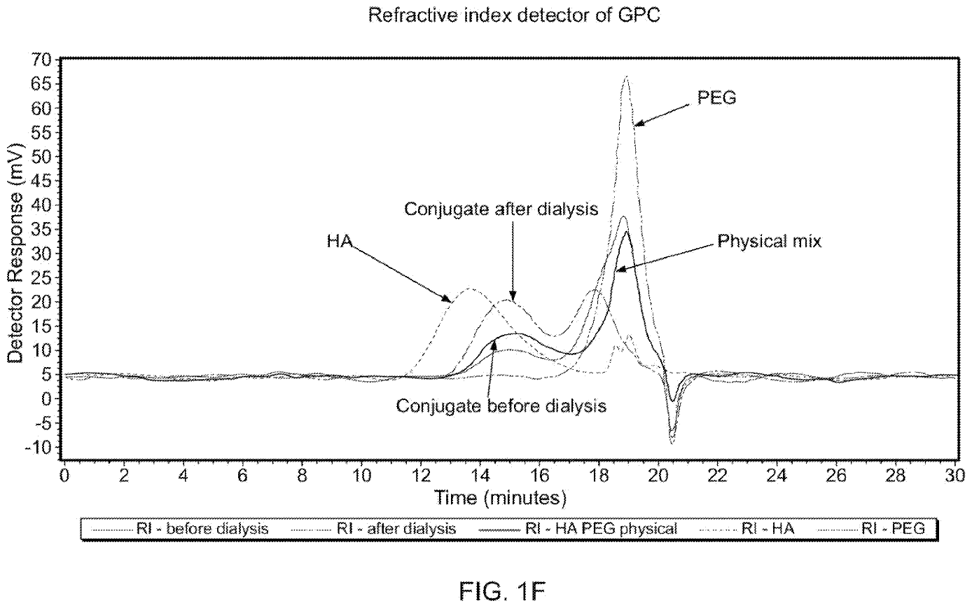

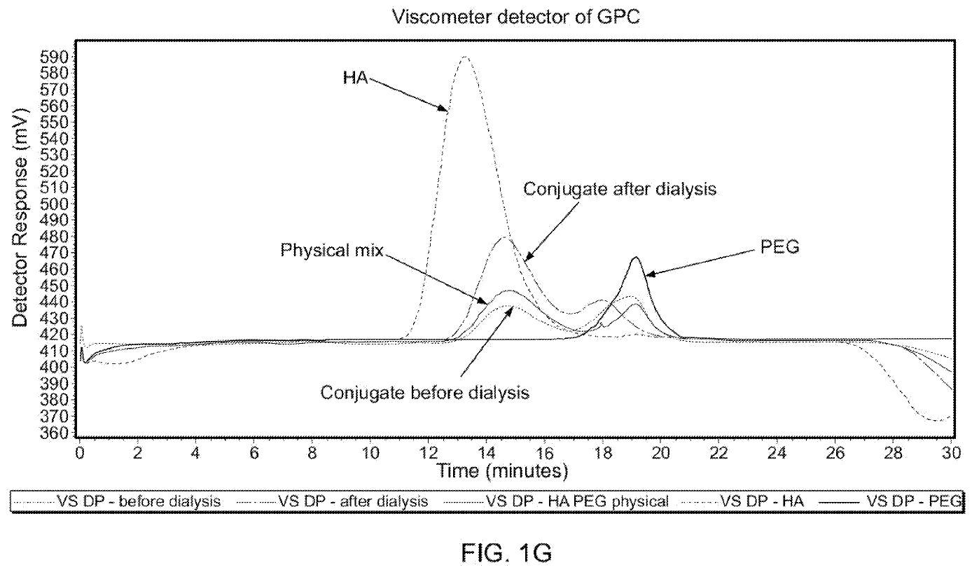

FIG. 1B shows a reaction scheme according to one non-limiting embodiment of the present disclosure. Aldehyde groups (—CHO) already present on HA react with the amino groups (—NH₂) in amino-PEG through a “Schiff-base” reaction to form a “Schiff-base” bond (—CH═N—).

### Results

FIG. 1C shows ¹H-NMR analyses of the structures of HA, PEG, and the HA-PEG conjugates were analyzed by ¹H-NMR and FTIR (FIGS. 1C and 1D, respectively). Table 1 below provides GPC analyses of molecular weight for the HA-PEG conjugates. Further GPC analyses of HA, PEG, and HA-PEG conjugates were conducted using light scattering detector (FIG. 1E), refractive index detector (FIG. 1F), and viscometer detector (FIG. 1G). Based on GPC analyses, HA-conjugate has a bimodal character of molecular weight. “Large MW” and “small MW” correspond to the “large” and “small” peaks (see FIGS. 1E-1G), respectively. Dialysis helps remove residual PEG

TABLE 1

| | | | | From |
|---|---|---|---|---|
| | Repeat 1 | Repeat | Average | manufacturer |
| dextran | 173,892 | 126,623 | 150,258 | 150k |
| HA | 37,926 | 31,619 | 34,773 | 32k-64k |
| PEG | 4,354 | 4,180 | 4,267 | 2k |
| Conjugate | 31,641 | N.A. | N.A. | N.A. |
| before | | | | |
| dialysis | | | | |
| large MW | | | | |
| Conjugate | 7,156 | N.A. | N.A. | N.A. |
| before | | | | |
| dialysis | | | | |
| small MW | | | | |
| Conjugate | 92,683 | 42,400 | 67,542 | N.A. |
| after | | | | |
| dialysis | | | | |
| large MW | | | | |
| Conjugate | 11,805 | 10,517 | 11,161 | N.A. |
| after | | | | |
| dialysis | | | | |
| small MW | | | | |

The refractive index of various HA-PEG conjugates was measured at 37° C. using an ABBMAT Automatic Refractometer (FIG. 1H).

The importance of this Example 1 is not only the creation of a material that achieves a specific index of refraction, but also the ability to chemically synthesize this material to achieve a range of specific index of refractions. In particular, the chemical synthesis method can be carried out to achieve specific index of refractions that are pre-defined. In addition, add mixtures of different HA-PEG conjugates of different ratios could be created to achieve specific index of refractions as necessary (e.g., in the clinic).

In addition, there is the possibility to cross-link the injected HA-PEG material once it has been injected within the corneal pocket. This cross-linking can be accomplished via photonic or chemical methods. This cross-linking can achieve chemical integration of the filler within the cornea as well as to stiffen the material within the corneal pocket.

To our best knowledge, using HA-PEG conjugates to adjust refractive index for ophthalmic applications have not previously been invented. In addition, predetermined properties of this HA-PEG conjugate match the needs for a corneal filler precisely.

The new procedure of “CoFi” is developed to correct hyperopia individually via minimally invasive means. A corneal filler is injected into a femtosecond laser-induced corneal pocket to increase the refractive power [Ref. 1.2]. 3D optical coherence tomography is used to gather precise data of the corneal shape. By using this imaging technology pre-operatively and postoperatively, the refraction change can be quantified and an individual adjustment using different filler volumes is possible. See FIG. 1I which shows refractive correction of a hyperopic eye. Farsightedness leads to blurred vision (eyeball is too short, placing the sharp image behind the retina). After filler injection into the femtosecond laser-created corneal pocket, the refractive power increases and the image falls onto the retina. The eye is emmetropic (right-sighted) [Ref. 1.2, 1.3].

It is important to determine an optimal balance between the flowability of the filler material for delivery via a fine gauge needle and the viscosity of the material so that it does not leak appreciable out of the corneal pocket following administration. This aspect of the material's viscosity can be optimized through several means, including but not limited to selection of different branched polymers via their branching (e.g., number of branches), selection of different branched polymers by their mass (e.g., total number of atoms in the composition), and selection of the two polymer masses and mass ratios. For example, a larger hyaluronic acid mass can be chosen which would increase the viscosity of the end material. Preliminary studies have indicated that, in a non-limiting example, that when using a small, four arm branched PEG and hyaluronic acid, that a hyaluronic mass before 750,000 daltons may be too low viscosity and a mass of 1,500,000 daltons may be too large.

Table 2 below provides a landscape in this field.

TABLE 2

| Procedure | Methodology | Characteristics |
|---|---|---|
| Ablative laser | Steepening the anterior corneal | Low predictability, serious |
| correction (PRK | curvature to increase refractive | complications, max. +3.0 dpt |
| and LASIK) | power by ablating peripheral | |
| | corneal areas | |
| Corneal filler | Filler injection into corneal | Biostable, |
| injection for | pocket cut by femtosecond | adjustable (up to +8.0 diopters) |
| hyperopia | laser | No removal of corneal tissue |

### Correction of Hyperopia by Large Area Filler Injection (CoFiH)

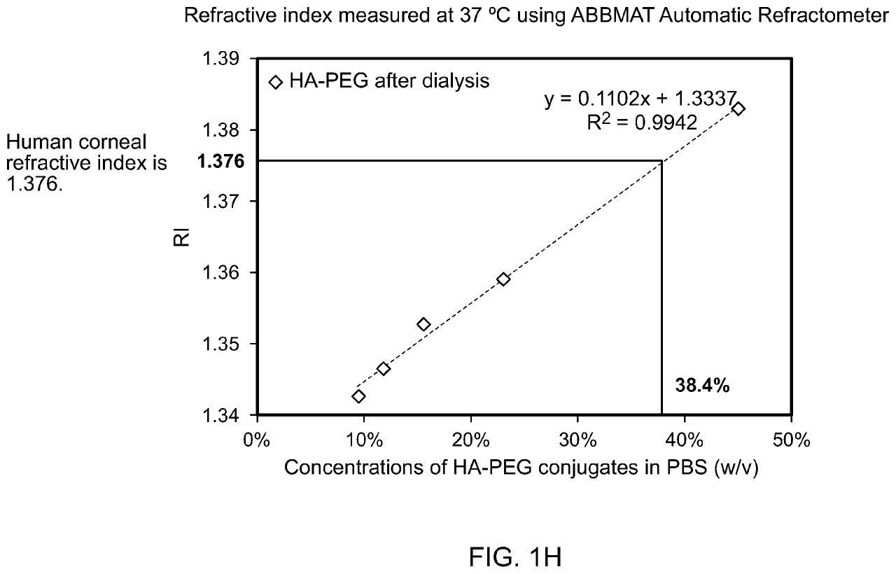

In the novel treatment of the method to correct hyperopia superficial large (diameter 6 mm) corneal pockets are cut with a femtosecond laser. A series of successful experiments on rabbit eyes ex vivo proved the concept by increasing the refractive power by up to 8 diopters depending on the filler volume. The procedure is adjustable as the filler volume can be individually adapted to the required refractive power change. See FIG. 2 which shows a refractive change after filler injection in two different depths. Stromal pockets in a depth of 120 μm lead to refractive power increases up to 8 diopters [see Ref. 1.6].

### Correction of Presbyopia by Central Filler Injection (CoFiP)

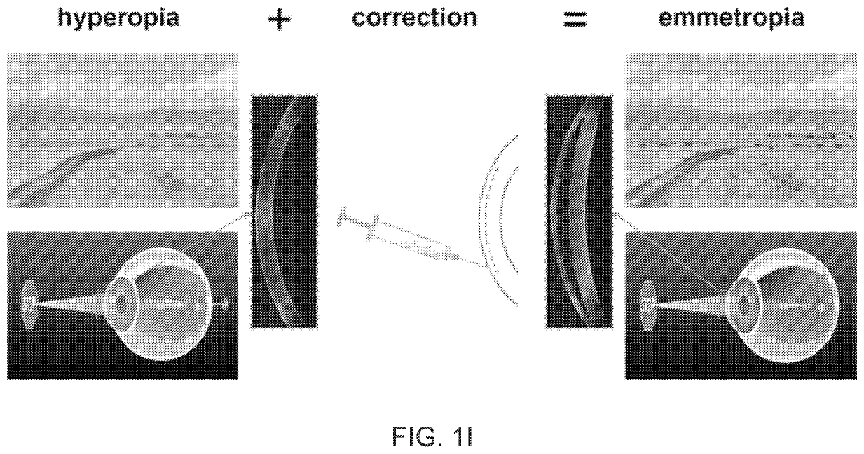

In the novel treatment of the method to correct presbyopia, deep and small (diameter of 2-3 mm) corneal pockets close to the posterior curvature are cut with a femtosecond laser. OCT images are used to determine the refraction changes. The flattening of the central posterior cornea increases the refractive power and thus creates a focus point for near vision. The peripheral cornea is kept unaffected for far vision. See FIG. 3 which shows a principle of presbyopia correction. Femtosecond laser-assisted, spherical corneal pockets close to the posterior curvature are established with a diameter of 2-3 mm. Injection of corneal filler leads to a central flattening of the posterior curvature and thus an increase of central refractive power.

A series of successful experiments on 57 rabbit eyes ex vivo proved the concept by increasing the central refractive power by up to 3.0±0.6 dpt depending on the filler volume.

Minor changes on the anterior curvature (0.43±0.50 dpt) and the peripheral posterior curvature (0.41±0.19 dpt) more or less neutralize each other so that only the change of the central posterior cornea remains providing a bi-focality, treating presbyopia.

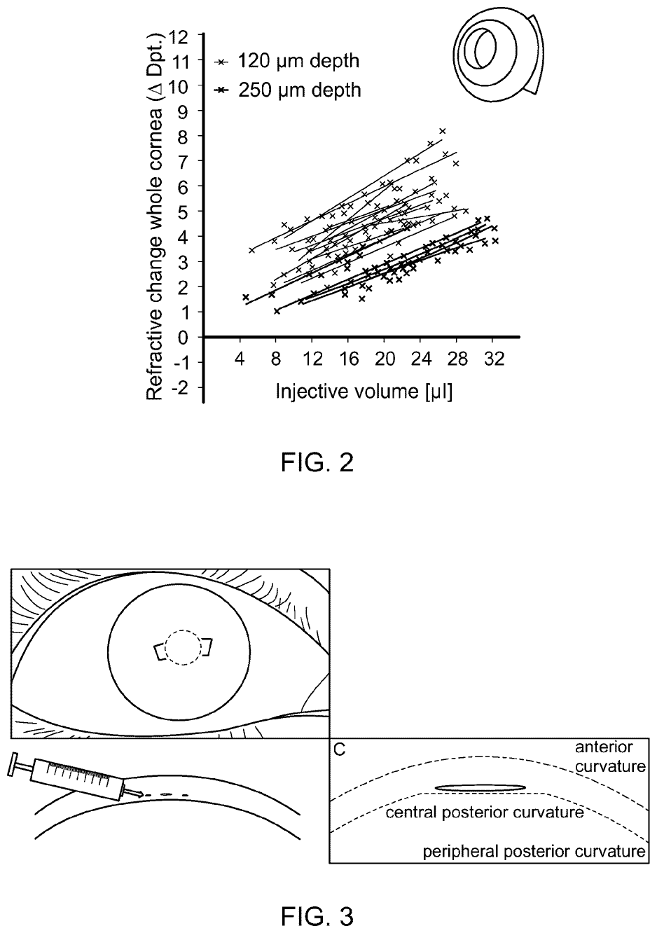

The procedure is adjustable as the filler volume can be individually adapted to the required refractive power change. See FIG. 4 which shows a refractive change after filler injection in two different depths. Stromal pockets in a depth of 120 μm lead to refractive power increases up to 8 diopters. [see Ref. 1.9].

### REFERENCES FOR EXAMPLE 1

1.1. Bope E T, Kellerman R D., Conn's Current Therapy, Philadelphia, PA: Saunders; 2013:323-326.
1.2. Freidank, S, Vogel, A, Anderson, R R, Birngruber, R, Linz, N, “Correction of hyperopia by intrastromal cutting and liquid filler injection”, J Biomed Opt 24 (5), pp. 1-7, 7,
1.3. https://plaintips.com/hyperopia (accessed on May 31, 2019)
1.4. Schiefer U, et al., “Refractive errors”, Dtsch Arztebl Int 2016; 113 (41): 693-702.
1.5. Anderson H A, et al., “Minus-lens-stimulated accommodative amplitude decreases sigmoidally with age: a study of objectively measured accommodative amplitudes from age 3”, Invest Ophthalmol Vis Sci 2008; 49:2919-2926.
1.6. Wertheimer, C M; Brandt, K; Kaminsky, S; Elhardt, C; Kassumeh, S A; Pham, L; Schulz-Hildebrandt, H; Priglinger, S; Anderson, R R; and Birngruber, R, “Refractive Changes After Corneal Stromal Filler Injection for the Correction of Hyperopia”, J Refractive Surg, pp. 406-413, 2020
1.7. Baily C, et al., “Preloaded refractive-addition corneal inlay to compensate for presbyopia implanted using a femtosecond laser: one-year visual outcomes and safety”, J Cataract Refract Surg 2014; 40:1341-1348.
1.8. Pinelli R, et al., “Correction of presbyopia in hyperopia with a center-distance, paracentralnear technique using the Technolas 217z platform”, J Refract Surg 2008; 24:494-500.
1.9. Kassumeh, S; Luther, J K; Wertheimer, C M; Brandt, K; Schenk, M S; Priglinger, SG; Wartak, A; Apiou-Sbirlea, G; Anderson, R R, and Birngruber, R., “Corneal Stromal Filler Injection as a Novel Approach to Correct Presbyopia—An Ex Vivo Pilot Study”, TVST, no. 9 (7), pp. 30-30, 2020

The citation of any document or reference is not to be construed as an admission that it is prior art with respect to the present invention.

### Example 2

### Background of Example 2

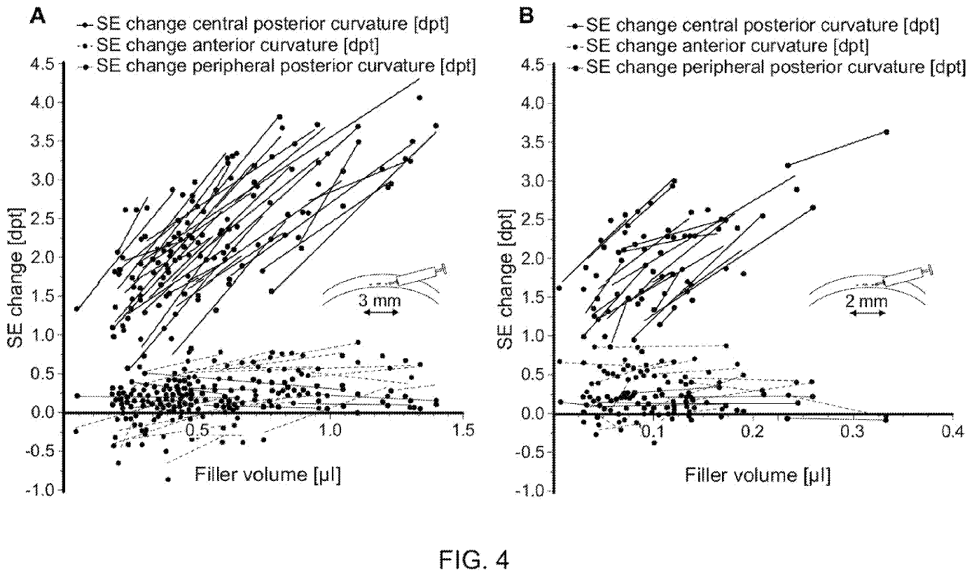

The Healthcare Challenge: Hyperopia, commonly referred to as farsightedness, is a common vision condition that affects a significant portion of the global population. [Ref. 2.1] It is a refractive error and occurs either because the eye's refractive power is weak due to the shape of the cornea (lens) inside the eye, or because the eyeball is too short. [Ref. 2.3] In hyperopia, the image of nearby objects becomes focused behind the retina, making objects up close appear out of focus (FIG. 5). According to the National Eye Institute, approximately 5-10% of Americans have hyperopia. [Ref. 2.2] Visual impairment caused by uncorrected hyperopia can significantly impact an individual's daily activities and overall quality of life. People with severe hyperopia may also experience symptoms such as eye strain, headaches, and fatigue.

The Existing Methods: Refractive surgery has been utilized to correct these refractive errors by reshaping the cornea's curvature. To correct hyperopia, the corneal surface needs to be steepened to increase the refractive power. However, despite the development of various surgical techniques, including hexagonal keratotomy, keratophakia, keratomileusis, and thermokeratoplasty, steepening the cornea remains challenging due to concerns related refractive result to predictability and potential complications. [Ref. 2.4, 2.5, 2.6] Hyperopic photorefractive keratectomy and hyperopic laser in situ keratomileusis (LASIK) are the established techniques in clinical use. [Ref. 2.7, 2.8] Using these techniques, the cornea is steepened through the ablation of the corneal stroma in a ring-shaped pattern, which decreases the radius of curvature in the central region and increases the refractive power. While these methods are safer and more effective than previous techniques, the results of hyperopic treatments are not as good as those of myopic treatments. It was shown that hyperopic correction with acceptable efficacy is only possible to approximately +4.0 diopters, whereas myopic correction can be achieved exceeding −11.0 diopters. Moreover, any error during the procedure can result in lifelong deficits in vision, as the corrective surgery cannot be undone. Hyperopia correction is associated with decreased result predictability and stability.

A new method involves altering the cornea's refractive power by introducing material directly into its stroma, changing its shape and thus focusing power. This technique eliminates the necessity for corneal steepening and offers potential reversibility. However, the hydrogel materials used during development were found not to be suitable for long-term use. [Ref. 2.9] Moreover, these hydrogels cause some serious issues including decentralization, peri-inlay deposits, and loss of more than 2 lines of vision, in the early postoperative period, limiting their applicability in clinical development. Thus, improving the composition of corneal filler materials is essential to make this approach viable.

The Need: A novel approach is needed, which facilitates: (i) refractive error correction without tissue removal, (ii) that preserves biomechanical stability, and (iii) allows for fine-tuning of the refractive result. To meet such requirements, we propose developing and injecting a novel filler material into a preformed intracorneal pocket. Compared with synthetic hydrogels, injection of viscous filler material is less likely to induce corneal decompensation by altering intracorneal nutrient diffusion, and re-injections allow the refractive result to be fine-tuned.

Permeable liquid materials, such as filler materials based on polysaccharide (PS), have undergone comprehensive investigation, and have shown successful applications in various other related biomedical fields, including dermatology and cataract surgery, over many years. [Ref. 2.10] Although these fillers might be suitable for intra-corneal injection, their large refractive index deviation from surrounding cornea limit their clinical applicability. [Ref. 2.11]

### Summary of Example 2

We propose to synthesize covalent conjugates of polyether (PE) and polysaccharide (PS) to address this gap. A PE moiety with different degrees of functionalization and/or molecular weights will be utilized to fine-tune the solubility, viscosity, and refractive index of the synthesized PE-PS conjugates. The material will be selected in such a way that its refractive index matches that of the corneal stroma in order to avoid optical inhomogeneities that could compromise vision acuity. The materials will also be chosen to be appropriately flowable so that it can be injected into the corneal pocket with a thin needle while maintaining enough viscosity to prevent leakage during the procedure.

### Preliminary Data

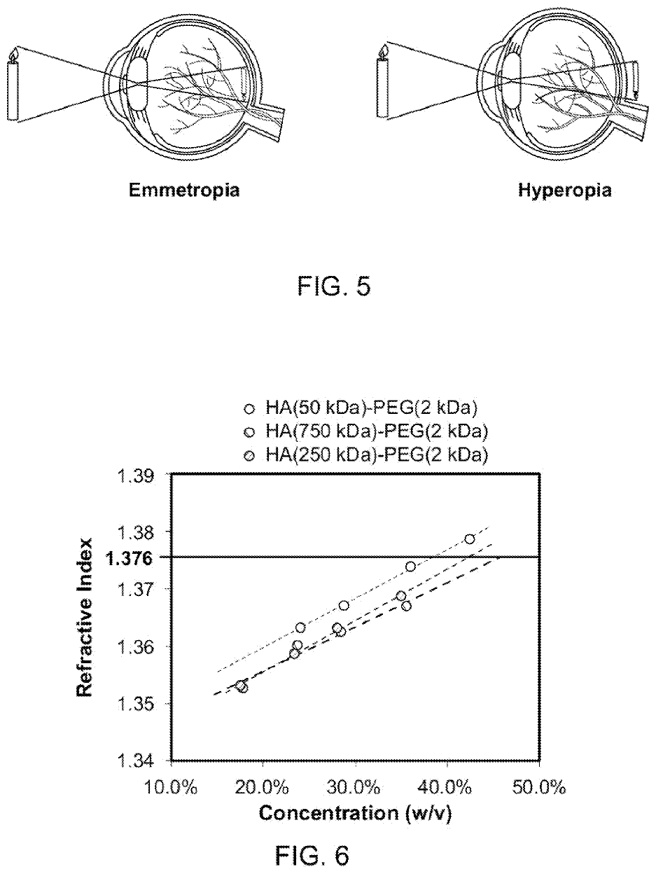

To evaluate the feasibility of this approach, preliminary studies were carried out. These initial investigations helped us in determining practicability and potential success of the idea. We prepared various hyaluronic acid (HA)-PEG conjugates by linking aldehyde groups of different HAs (50, 250, and 750 kDa) to amino groups of 4-arm PEG-amine (2 kDa). [Ref. 2.12, 2.13] The conjugates were characterized by their nuclear magnetic resonance (NMR), gel permeation chromatography (GPC), and Fourier-transform infrared spectroscopy (FTIR) techniques. Different concentrations of the synthesized conjugates were prepared, and their refractive index was measured at 33° C. (corneal temperature) (see FIG. 6).

These tests resulted in materials that could readily be tuned across a wide range of viscosities and indices of refraction via the choice of the HA molecular weight and stoichiometric ratio of HA to PEG. An empirical equation was developed to determine the chemical reagents and reaction conditions to create filler materials that match the refractive index of the cornea (n=1.376).

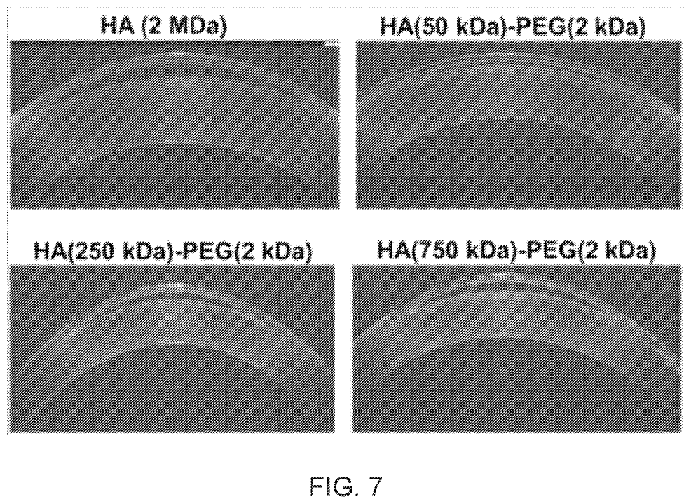

Materials of different viscosity with a refractive index of 1.376 were then injected ex vivo into rabbit eye cornea and their effect on the cornea was investigated (FIG. 7). The results were compared with a reported 2 MDa HA (1% w/v), which had a lower density than the HA-PEG conjugate described herein. The 2 MDa HA material possesses the appropriate viscosity for injection and good stability in the stromal pocket, [Ref. 2.11, 2.14, 2.15] however, its index of refraction does not match with that of the corneal stroma, thus the clinical applicability is still elusive, remaining a significant bottleneck. Through a careful design, we tried to overcome this challenge by developing a covalent conjugate of HA and PEG. We have chosen 2 kDa 4-arm-PEG-amine and linear amine-mPEGs for conjugation with HA backbone because of their excellent water solubility, cytocompatibility, scalability, and good in vivo clearance. Two synthesized test materials, i.e., HA (50 kDa)-PEG (2 kDa) and HA (250 kDa)-PEG (2 kDa) were found to be less viscous at the concentration required to match the refractive index and resulted in immediate leakage from the stromal pocket. The results indicated that the higher molecular weight HA (750 kDa)-PEG (2 kDa) was able to achieve the correct index of refraction, and showed a degree of retention in the stromal pocket (FIG. 7), comparable to HA (2 MDa). Higher analogs of HA (i.e., 1.0 MDa, 1.5 MDa, and 2.5 MDa) in combination with different PEG, mPEG, and higher analogue PEGs may further improve performance with both appropriate viscosity and uniform distribution throughout the cornea for optimum hyperopia correction.

### Potential Scientific and Medical Impact

We envision our developed material will address the need for personalized hyperopia treatment by performing a refractive correction through corneal shape adjustment. The injection of the filler material into the intrastromal pocket is additive and does not require tissue removal by excimer laser ablation. Therefore, it does not weaken the mechanical stability of the cornea as LASIK does. Moreover, the proposed injectable corneal filler holds the promise of providing a secure and efficient approach to treating hyperopia at any stage in a minimal invasive way. The project has been carefully designed around a set of common intermediates from which a library of materials can be readily generated in a minimum number of steps. Given that the type of intermediate (polysaccharides and polyethers) used have already been approved by the United States Food and Drug Administration (FDA), the chance for negative side effects is minimized due to their biocompatible profiles. There is also a potential economic benefit as many intermediates are available at low cost, which enhances future translation and commercialization. Moreover, the investigation of the filler material and its appropriateness towards hyperopia will lead to a deeper understanding of the physicochemical properties of the material and this knowledge would not only be helpful for the hyperopia application but also for other applications in refractive surgery such as presbyopia, in dermatology and cataract surgery.

The potential implications of this innovation are vast, allowing for the addition or removal of filler material in cases requiring fine refinement of refractive power or compensating for regression. For all the above-mentioned reasons, we truly believe that this project is fascinating and has potentially far-reaching research and medical impacts that will benefit patients worldwide. Moreover, the filler we propose to develop will be affordable and can be developed on a large scale. In our pursuit of potentially commercializing this material, we are proactively seeking the best-fit industrial partners to facilitate the transition from our research lab into medical product.

### REFERENCES FOR EXAMPLE 2

2.1. Holden, B. A.; Fricke, T. R.; Ho, S. M.; Wong, R.; Schlenther, G.; Cronjé, S.; Burnett, A.; Papas, E.; Naidoo, K. S.; Frick, K. D., “Global vision impairment due to uncorrected presbyopia”, Arch. Ophthalmol. 2008, 126, 1731-1739.
2.2. National Eye Institute. https://www.nei.nih.gov/learn-about-eye-health/eye-conditions-and-diseases/farsightedness-hyperopia (accessed on Feb. 28, 2023).
2.3. Hashemi, H.; Fotouhi, A.; Yekta, A.; Pakzad, R.; Ostadimoghaddam, H.; Khabazkhoob, M., “Global and regional estimates of prevalence of refractive errors: Systematic review and meta-analysis”, J. Curr. Ophthalmol. 2018, 30, 3-22.
2.4. Basuk, W. L.; Zisman, M.; Iii, G. O. W.; Wilson, L. A.; Binder, P. S.; Thompson, K. P.; Grossniklaus, H. E.; Stulting, R. D., “Complications of hexagonal keratotomy”, Am. J. Ophthalmol. 1994, 117, 37-49.
2.5. Friedlander, M. H.; Werblin, T. P.; Kaufman, H. E.; Granet, N. S., “Clinical results of keratophakia and keratomileusis”, Ophthalmology 1981, 88, 716-720.
2.6. Feldman, S. T.; Ellis, W.; Frucht-Pery, J.; Chayet, A.; Brown, S. I., “Regression of effect following radial thermokeratoplasty in humans”, J. Refract. Surg. 1989, 5, 288-291.
2.7. Pallikaris, I. G.; Panagopoulou, S. I., “PresbyLASIK approach for the correction of presbyopia”, Curr. Opin. Ophthalmol. 2015, 26, 265-272.
2.8. Settas, G.; Settas, C.; Minos, E.; Yeung, I. Y., “Photorefractive keratectomy (PRK) versus laser assisted in situ keratomileusis (LASIK) for hyperopia correction”, Cochrane Database Syst. Rev. 2012 (6), CD007112 (2012).
2.9. Mulet, M. E.; Alio, J. L.; Knorz, M. C., “Hydrogel intracorneal inlays for the correction of hyperopia: outcomes and complications after 5 years of follow-up”, Ophthalmology 2009, 116, 1455-1460.
2.10. Tezel, A; Fredrickson, G. H., “The science of hyaluronic acid dermal fillers”, J. Cosmet. Laser Ther. 2008, 10, 35-42.
2.11. Freidank, S.; Vogel, A.; Anderson, R. R.; Birngruber, R.; Linz, N., “Correction of hyperopia by intrastromal cutting and liquid filler injection”, J. Biomed. Opt. 2019, 24, 058001.
2.12. Tan, H.; Chu, C. R.; Payne, K. A.; Marra, K. G., “Injectable in situ forming biodegradable chitosan-hyaluronic acid based hydrogels for cartilage tissue engineering”, Biomaterials 2009, 30, 2499-2506.
2.13. Fan, M.; Zhang, Z.; Mao, J.; Tan, H., “Injectable multi-arm poly(ethylene glycol)/hyaluronic acid hydrogels for adipose tissue engineering”, J. Macromol. Sci., Part A 2015, 52, 345-352.
2.14. Wertheimer, C. M.; Brandt, K.; Kaminsky, S.; Elhardt, C.; Kassumeh, S. A.; Pham, L.; Schulz-Hildebrandt, H.; Priglinger, S.; Anderson, R. R.; Birngruber, R., “Refractive changes after corneal stromal filler injection for the correction of hyperopia”, Journal of Refractive Surgery 2020, 36, 406-413.
2.15. Kassumeh, S.; Luther, J. K.; Wertheimer, C. M.; Brandt, K.; Schenk, M. S.; Priglinger, S. G.; Wartak, A.; Apiou-Sbirlea, G.; Anderson, R. R.; Birngruber, R., “Corneal stromal filler injection as a novel approach to correct presbyopia—An ex vivo pilot study”, Translational Vision Science & Technology 2020, 9, 30-30.

The citation of any document or reference is not to be construed as an admission that it is prior art with respect to the present invention.

### Example 3

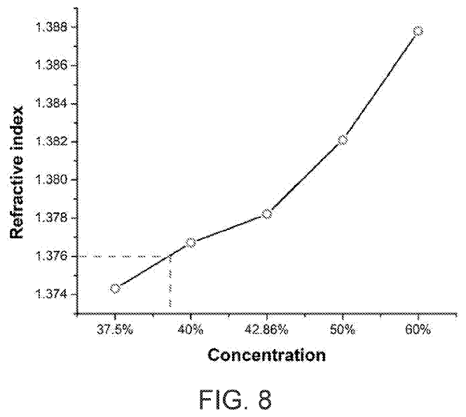

HA-4-PEG and HA-mPEG conjugates were prepared by the process as described herein and shown in Schemes 1 and 2, respectively. The refractive index of HA-mPEG at 33° C. is shown in FIG. 8.

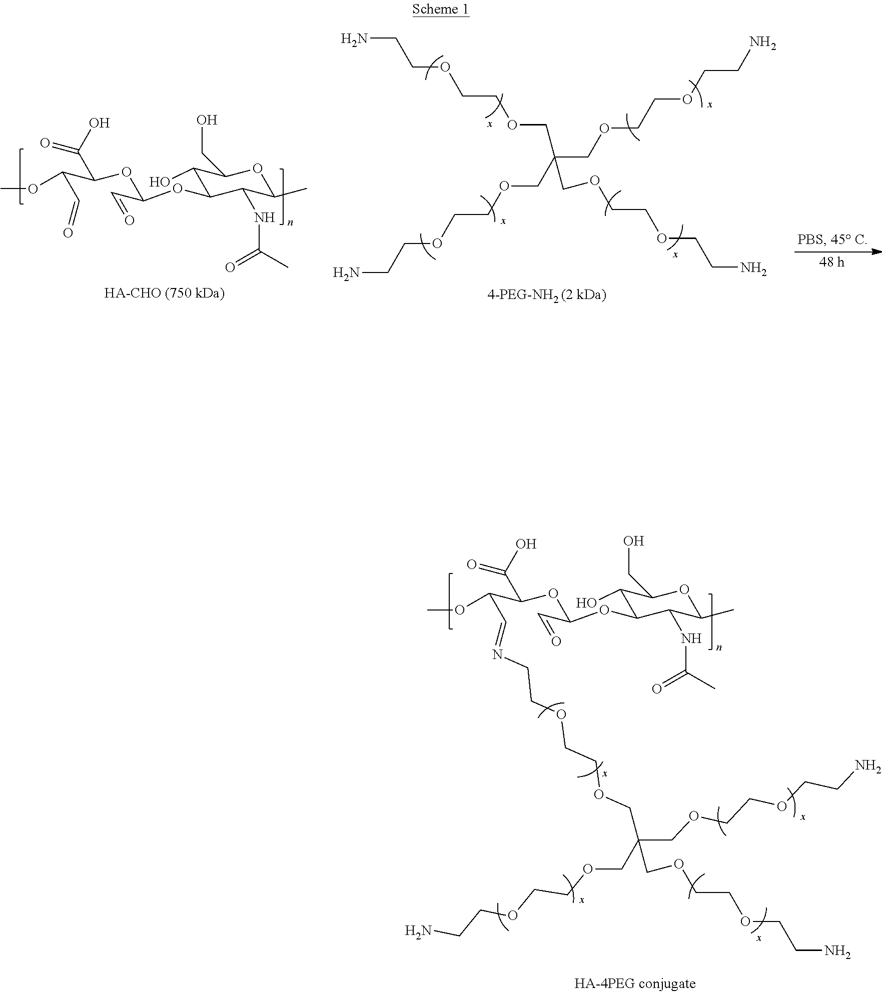

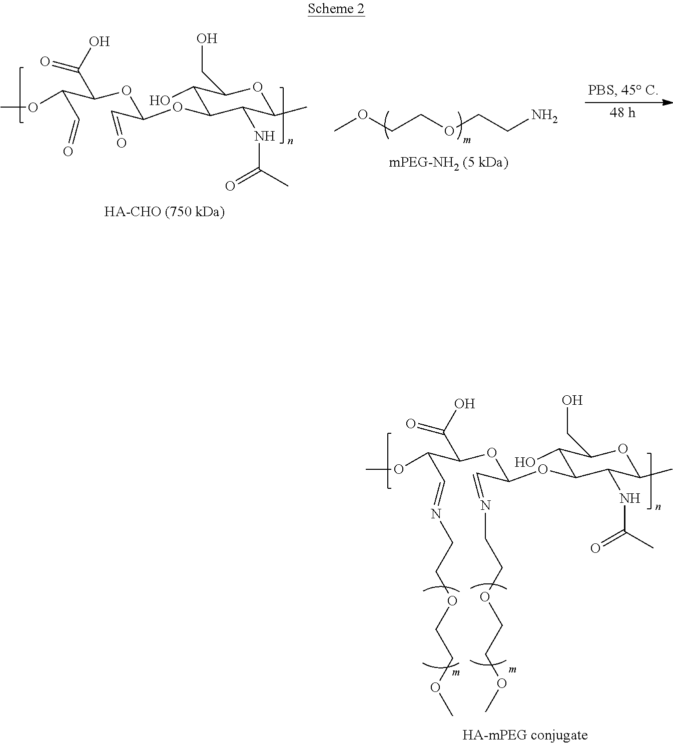

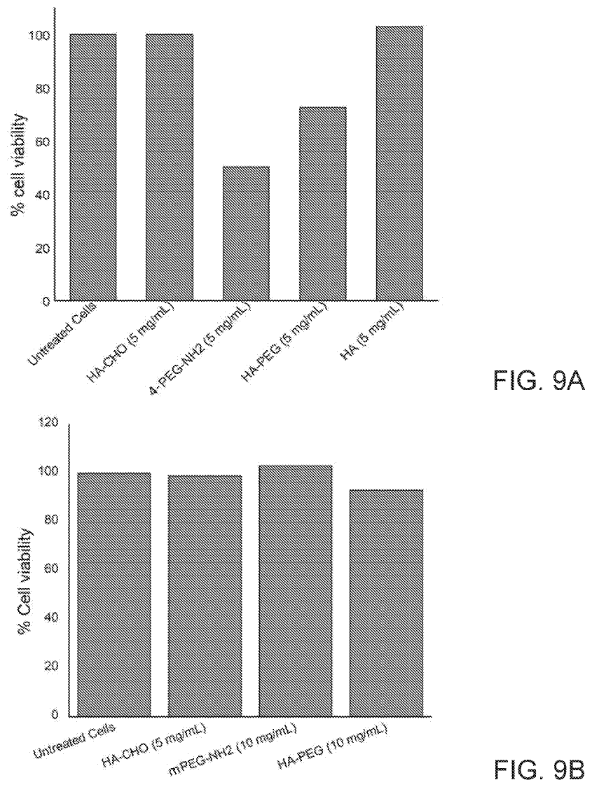

In vitro cell viability of the corneal filler material was assessed using MTT assays. Briefly, NIH3T3 cells were seeded in a 96-well plate (~10000 cells/well in 100 μL DMEM cell medium) and cultured at 37° C. overnight. Subsequently, the cell medium was aspirated from all the wells and replaced with filler material diluted in DMEM at various concentrations. For background subtraction, wells containing only filler material in medium without cells were utilized. The cells were then incubated for 24 h at 37° C. before removing the material from all the wells. MTT solution (5% in DMEM) was added and incubated for 4 h to facilitate the formation of MTT-formazan crystals. Following this, the cell medium was aspirated, and DMSO was added to dissolve the MTT-formazan crystals. Absorbance was recorded at 570 nm using a microplate reader, where absorbance is proportional to the number of viable cells. The cell viability was determined by adjusting the non-treated cells to 100%. The MTT assay results for HA-4-PEG and HA-mPEG conjugates are shown in FIG. 9A and FIG. 9B, respectively. The observed cytotoxicity of HA-4PEG (FIG. 9A) might be due to the presence of multiple amino groups or unreacted 4-PEG-NH2 in the HA-4PEG conjugate material. HA-mPEG, on the other hand, was well tolerated in the cellular assay (FIG. 9B).

Thus, the present invention provides compositions, systems, and methods for treating hyperopia and presbyopia without tissue removal to increase the refractive power of the cornea.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another.

Although the invention has been described in considerable detail with reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A polymer conjugate, or a pharmaceutically acceptable salt thereof, comprising hyaluronic acid repeating units of formula (I) and a plurality of aldehyde units of formula (II) 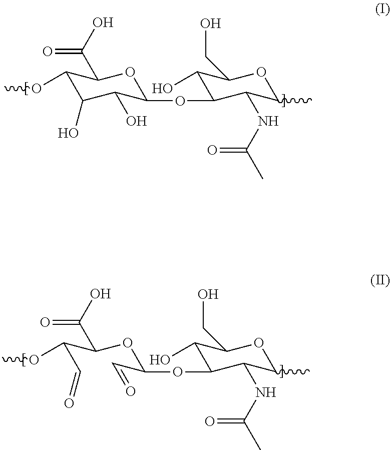
wherein
at least one —C(O)H group in the plurality of aldehyde units is replaced by —CH═N—R; and
R is a polyether group.

2. The polymer conjugate of claim 1, or a pharmaceutically acceptable salt thereof, which consists of hyaluronic acid repeating units of formula (I) and the plurality of aldehyde units of formula (II), wherein at least one —C(O)H group in the plurality of aldehyde units is replaced by —CH═N—R.

3. The polymer conjugate of claim 1, or a pharmaceutically acceptable salt thereof, wherein the polyether group comprises a polyethylene glycol (PEG) group, a polyglycerol group, a pluronic group, or a combination thereof.

4. The polymer conjugate of claim 1, or a pharmaceutically acceptable salt thereof, wherein R has a terminal group R¹, and wherein R¹ is H, C₁₋₄ alkyl, OH, NH₂, C₁₋₄ hydroxyalkyl, C₁₋₄ aminoalkyl, O—C₁₋₄ alkyl, or NH—C₁₋₄ alkyl.

5. The polymer conjugate of claim 1, or a pharmaceutically acceptable salt thereof, wherein R is a multi-arm polyether group.

6. The polymer conjugate of claim 1, or a pharmaceutically acceptable salt thereof, wherein R is a 4-arm polyether group of —(CH₂CH₂O)_{y}CH₂C[CH₂(OCH₂CH₂)_{y}R¹]₃, and wherein y is 5-120.

7. The polymer of claim 6, or a pharmaceutically acceptable salt thereof, wherein R¹ is H, —OH, or —NH₂.

8. The polymer conjugate of claim 1, or a pharmaceutically acceptable salt thereof, wherein R is —(CH₂CH₂O)ₜ—R¹, and wherein t is 20-500.

9. The polymer of claim 8, or a pharmaceutically acceptable salt thereof, wherein R¹ is H, C₁₋₄ alkyl, —CH₂CH₂OH, or —CH₂CH₂NH₂.

10. The polymer conjugate of claim 1, or a pharmaceutically acceptable salt thereof, having an average molecular weight of about 50 kDa to about 5000 kDa.

11. The polymer conjugate of claim 1, or a pharmaceutically acceptable salt thereof, having an average molecular weight of about 500 kDa to about 2000 kDa.

12. The polymer conjugate of claim 1, or a pharmaceutically acceptable salt thereof, which is crosslinked.

13. The polymer conjugate of claim 1, or a pharmaceutically acceptable salt thereof, having a refractive index of about 1.3 to about 1.5.

14. The polymer conjugate of claim 1, or a pharmaceutically acceptable salt thereof, having a viscosity of about 5,000 cP to about 130,000 cP.

15. A polymer conjugate, comprising an aldehyde hyaluronic acid conjugated with a polyether having a terminal amino group, whereby a —CH═N— bond is formed between an aldehyde group of the aldehyde hyaluronic acid and the terminal amino group of the polyether.

16. (canceled)

17. (canceled)

18. (canceled)

19. (canceled)

20. (canceled)

21. (canceled)

22. A method of preparing a polymer conjugate, the method comprising:
reacting an aldehyde hyaluronic acid comprising hyaluronic acid repeating units of formula (I) and a plurality of aldehyde units of formula (II) with a polyether having a terminal amino group, 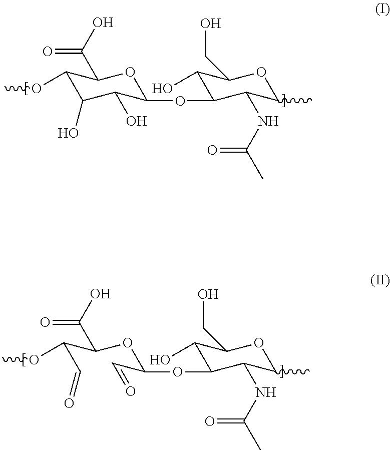
whereby at least one —C(O)H group in the plurality of aldehyde units forms a —CH═N— bond with the terminal amino group of the polyether to produce the polymer conjugate.

23. (canceled)

24. (canceled)

25. (canceled)

26. (canceled)

27. A polymer conjugate produced by the method of claim 22.

28. (canceled)

29. (canceled)

30. An injectable filler material comprising the polymer conjugate of claim 1, or a pharmaceutically acceptable salt thereof, and at least one additional component.

31. (canceled)

32. (canceled)

33. (canceled)

34. (canceled)

35. (canceled)

36. A method for treating an ametropia in a patient, the method comprising:
injecting a filler material comprising the polymer conjugate of claim 1 into the patient's cornea in an amount sufficient to steepen the anterior surface and flatten the posterior surface of the cornea to increase the refractive power of the cornea by a predetermined correction due to changing both surfaces of the cornea,
wherein the polymer conjugate forms a corneal implant with a lenticular shape within the cornea.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A method to treat presbyopia in a patient, the method comprising: creating an optical bifocality by creating a small incision (2-3 mm in diameter) in the optical center of the patient's cornea deep enough (about 50-150 μm away from the posterior surface) to selectively flatten the central part of the posterior surface of the cornea, increasing only the central refractive power by 1-3 diopters but leaving the peripheral refraction unchanged.

50. (canceled)

51. (canceled)

52. (canceled)

53. (canceled)
